(19) United States
(12) Patent Application Publication
(10) Pub. No.: US 2013/0231582 A1
(43) Pub. Date: Sep. 5, 2013

(54) CONTINUOUS HEALTHCARE MONITORING CONCIERGE

(71) Applicant: Manoj K. Prasad, Bloomfield Hills, MI (US)

(72) Inventor: Manoj K. Prasad, Bloomfield Hills, MI (US)

(73) Assignee: XpertEase Consultants, LLC, Bloomfield Hills, MI (US)

(21) Appl. No.: 13/586,467

(22) Filed: Aug. 15, 2012

Related U.S. Application Data
(60) Provisional application No. 61/606,061, filed on Mar. 2, 2012.

(51) Int. Cl. A61B 5/00; A61B 10/00

(52) U.S. Cl. 600/551; 600/300

(57) ABSTRACT

A system and method for monitoring a health parameter of a vehicle occupant where an occupant profile is created that identifies a biological parameter to measure, a measure health parameter to report on and corresponding thresholds that trigger an alert. A vehicle has an integrated sensor that measures the biological parameter according to the profile and provides a signal of the measured biological parameter. A computing device records the measured biological parameter and derives the measured health parameter from the measured biological parameters. If the measured health parameter passes a threshold, then an alert is triggered, and the occupant or interested party is notified.

### CROSS REFERENCE TO RELATED APPLICATIONS

This application claims the benefit of the priority date of U.S. Provisional Patent Application Ser. No. 61/606,061, titled “Continuous Healthcare Monitoring Concierge,” filed Mar. 2, 2012.

### BACKGROUND OF THE INVENTION

1. Field of the Invention

This invention relates generally to a system and method for personal health monitoring and, more particularly, to a system and method for establishing an occupant profile, gathering personal biological data over time from the occupant based on the profile when he/she is seated in a vehicle, storing the data in a central data repository, analyzing the data and sending notifications based on the data.

2. Discussion of the Related Art

Coupling the government's attempts to make the entire population insurable with the population's increasing age and the fact that new healthcare services are constantly becoming available, an unprecedented increase in demand for healthcare services is being created. The existing shortage of trained healthcare providers and the projected increase in the gap for the foreseeable future are compounding the problem. As if these challenges were not enough, the evolving entertainment media is creating incessant pressure on individuals to monitor and maintain good health, which has convinced an increasing number of people to seek out and obtain healthcare services contributing to the runaway cost for providing healthcare.

The insurance industry, the Institute of Medicine, Centers for Medicare and Medicaid and most other health organizations all agree that popularizing preventive medicine, rather than curative medicine, will result in a healthier population that, in the long run, would require less healthcare, but may allow people to live longer, resulting in a slight decrease in the net demand for healthcare. This projected reduction is not enough to offset the increased demand. Hence, there is a need to leverage modern technology to create tools to automate some of the processes required in the provisioning and maintenance of healthcare and provide tools to individuals to monitor their own health.

Commuting is an essential and growing component of the daily lives of most American workers making up over 23% of all trips taken, which is a significant percentage of the lives of a large population segment. In 2009, of the 140 million workers in the United States, 86.1% commuted in a car, truck, bus or van, 76.1% drove alone and another portion of people commuted by air. Commuting has been shown to correlate with several health problems, but detailed studies have not been done to fully understand the association.

A need exists for a health monitoring system and method that enables the monitoring of health parameters of an individual as he/she commutes to utilize the commute time efficiently.

### SUMMARY OF THE INVENTION

In accordance with the teachings of the present invention, a system and method for monitoring a health parameter of a vehicle occupant is disclosed. An occupant profile is created that identifies a biological parameter to measure, a measure health parameter to report on and corresponding thresholds that trigger an alert. A vehicle has an integrated sensor that measures the biological parameter according to the profile and provides a signal of the measured biological parameter. A computing device records the measured biological parameter and derives the measured health parameter from the measured biological parameters. If the measured health parameter passes a threshold, then an alert is triggered and the occupant or interested party is notified.

Additional features of the present invention will become apparent from the following description and appended claims, taken in conjunction with the accompanying drawings.

### BRIEF DESCRIPTION OF THE DRAWINGS

FIG. 1 is a drawing of a first embodiment of a health-monitor concierge with a dedicated display screen; and

FIG. 2 is a block diagram of a second embodiment of the health-monitor concierge with a central data repository and a monitoring station.

### DETAILED DESCRIPTION OF THE EMBODIMENTS

The following discussion of the embodiments of the invention directed to a system and method for continuously monitoring health parameters is merely exemplary in nature, and is in no way intended to limit the invention or its applications or uses.

### Profile Creator

A profile creator is part of a health-monitor concierge that is located in a vehicle and monitors the health parameters of a passenger of the vehicle. The health-monitor concierge uses the profile creator to generate an occupant profile that will inform the health-monitor concierge what to monitor, when and how to send notification alerts and other configuration information. The profile creator will go through a registration process that can be very simple or more involved with a series of questions. A simple profile creation process would be recording when an occupant sits in a vehicle seat. A more involved profile creation process would gather information about the occupant's contact information, height, weight, age, medical concerns, personal physician, health parameters to be measured, what monitoring to do (thresholds that send off notifications) and other information. The information could come from the occupant, a physician, a nurse, a caregiver, another computer system or any person who can answer the questions needed to complete the profile.

A health-monitor concierge uses the profile to know what health parameters to monitor. In the profile creation process, which is a registration process, the occupant could identify their particular health concerns and the health-monitor concierge would then know the set of health parameters to monitor. The profile creator can present the occupant with a list of medical concerns that can be monitored. The profile creator could have a list of medical concerns or conditions, and for each medical concern the profile creator would have a list of biological parameters to measure, health parameters to monitor and a set of pre-programmed activities to lead the occupant through. For example, the occupant could have concerns about sleep apnea in which case the health-monitor concierge could recommend monitoring oxygen concentrations and provide instructions on proper sleep techniques. Similarly, different conditions (for example epileptic conditions, obesity or high blood pressure) could have a specific set of health parameters to monitor and activities that the health-monitor concierge could do for the occupant to address the health concerns like educational material or biofeedback.

The health-monitor concierge can have monitoring thresholds customized by the occupant or other interested parties to desired settings to create pre-established thresholds that if passed cause an alert.

The profile can include information about how to notify the interested parties about alerts. The profile can specify if the collected data should be made available to the occupant or other interested parties on a personal webpage. The profile can include if and where to publish the measured health parameters. For example, the data could authorize sending the gathered data to a website, where that site can combine the gathered data with other exercise, nutrition and diet information. The profile could authorize the data be sent to a social web site like Facebook.

The health-monitor concierge could have various thresholds, and limits customized for the occupant's individual needs that if crossed would send off notifications. For example, if the medical concern is high blood pressure, a first occupant may want an alert if the blood pressure exceeds 130/90 (perhaps as instructed by their physician). A different occupant's profile might be monitoring whether the blood pressure exceeds 120/80. Another example of monitoring could be monitoring an occupant who has a heart condition that causes a missed beat every 10 heartbeats, on average. In this case, the threshold could be if the occupant—on average—misses more than 1-in-10 heartbeats trigger an alert. For blood alcohol level, perhaps one occupant's profile might require an alert be sent if the blood alcohol level reaches 0.05%, while another occupant profile might have the threshold be 0.08%. If the concern is being overweight then, if the occupant is a member of Weight Watchers the health-monitor concierge could send Weight Watchers the occupant's weight on a periodic basis. The health-monitor concierge could provide feedback and encouragement based on the weight detected while the occupant is in the seat.

If Personally Identifiable Information, PII, is part of the profile, then the profile data may need encryption to be incompliance with the legal requirements. The need for encryption may be avoidable by using a unique identifier that the occupant remembers and is not associated to PII.

### Embodiment One

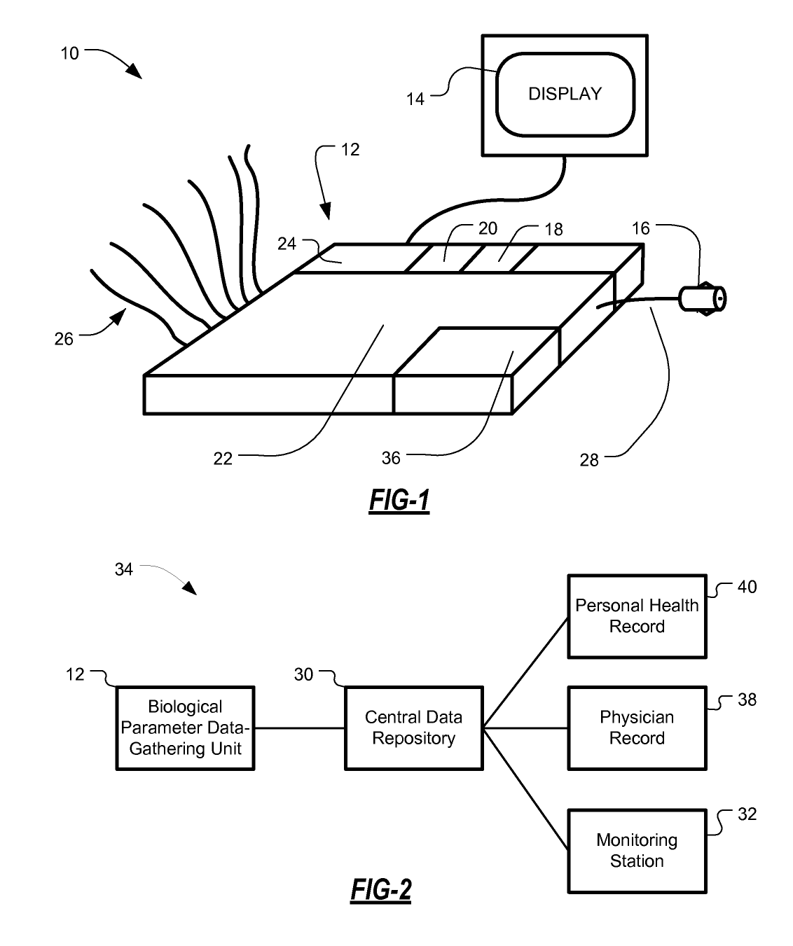

FIG. 1 is a drawing of a first embodiment of a health-monitor concierge 10 that has a dedicated display 14 and a biological parameter data-gathering unit 12 with a power pack 16, a short-range communication device 18, a long-range communication device 20, a computing device 22, a memory device 36, a power cord 28 and a display interface 24. The power pack 16 powers the data-gathering unit 12 and is charged via the power cord 28. The data-gathering unit 12 can have a back-up battery, not shown. The data-gathering unit 12 receives sensor signals from various sensors that provide signals of measured biological parameters. The sensor signals can be received by wires 26 or wirelessly by the short-range communication device 18. The measured biological parameters can be stored in the memory device 36. The computing device 22 uses the measured biological parameters to calculate monitored health parameters and displays them to the occupant by sending them to the display 14 through the display interface 24.

The communication devices 18 and 20 may be optional if the monitored health parameters are displayed on the display 14. Either one or both of the communication devices 18 or 20 can be present on the data-gathering unit 12. The short-range communication device 18 can be USB, Cat 5 (or another appropriate hard-wired connection), Bluetooth, infrared, Wi-Fi (IEEE 802.11), Apple AirPort wireless or other suitable short-range wireless communication means. The intent of the short-range communication device 18 is to allow the data-gathering unit 12 to gather sensor data and display, communicate and get feedback from the occupant though a computing device. The long-range communication device can be cell phone technology, Telematic communication, Wi-Fi or another appropriate technology.

The data-gathering unit 12 displays monitored health parameters to the occupant. The monitored health parameters are the measured biological parameters and other health parameters that the computing device 22 calculates from the measured biological parameters and profile information. The health-monitor concierge 10 can display the monitored health parameters and alerts to the occupant via the dedicated display 14 or could use the short-range communication unit 18 to send the data to another display device. For example, the other display device could be a cell phone, a vehicle infotainment screen, a GPS device screen (as offered by TomTom, Garmin or Magellan), a smart-phone device (communicating to with the short-range communication unit 18 that can be mounted in the vehicle), the dedicated display 14 or other suitable device that can display information to the occupant.

The health-monitor concierge 10 could communicate auditory alerts or other messages with a dedicated speaker and microphone, not shown, or by using a physical connection or the short-range communication unit 18 to send auditory messages to and from a vehicle audio system, a GPS device, a cell phone, a smart phone, a personal music player or any other device capable of producing sound for the occupant to hear to provide audio communication, such as audible alerts or two-way voice communication.

The data-gathering unit 12 can gather data on multiple biological parameters from an occupant of a vehicle. The data-gathering unit 12 collects the data and can calculate health parameters and can analyze the data in the computing device 22 and displays the health parameters in accordance with the occupant profile.

The health-monitor concierge 10 can be a freestanding unit that is self-contained with the built-in long-range communication device 20 and a built-in display.

The memory device 36 can store the occupant profile. The profile creation can be done using computer instructions stored in the memory device 26 or other non-volatile memory and run on the computing device 22 utilizing the dedicated display 14, or the profile creation could be run on a separate computer and the profile loaded in the memory device 26.

The data-gathering unit 12 can be mounted in a vehicle, where the vehicle can be a car, truck, bus, aircraft, mass transit or other transportation.

The health-monitor concierge 10 can work to improve the occupant's health. If the measured health parameters indicate the occupant is stressed, then the health-monitor concierge 10 could lead the occupant through a relaxation technique and monitor the biological parameters, and/or provide biofeedback to try to reduce the occupant stress response by providing biofeedback on how the occupant is doing at reducing their stress. If the occupant is a driver, then audio communication could be used. If the occupant is not a driver, then the display on the device could be used to also provide biofeedback.

Numerous sensors (not shown) can be mounted to various parts of the vehicle (seats, belts, steering wheels, gear shift handle, central console, dash board, seat handles etc.) to sense the various biological parameters of the occupant. These sensors can be connected by wires 26 or through the short-range communication device 18 to the data-gathering unit 12.

The data-gathering unit 12 can record the various biological parameters of the seat's occupant, such as weight, height, cardiac signals, respiration, temperature, oxygen saturation levels, blood pressure, blood sugar, blood alcohol, brain activity, and other biological parameters.

A weight sensor can measure the occupant's weight by measuring the weight below the seat, for example, at the seat attachments.

A height sensor can report on the seat placement, which the health-monitor concierge 10 can use to infer the height of the occupant.

A cardiac sensor can measure aspects of the heart's electrical activity. The heart's electrical activity can be obtained in different ways. For example, active monitoring could occur by having the occupant wear stick-on sensors. Passive monitoring can occur by using ultra-sensitive sensors mounted on seat belts, the steering wheel, seat handles, other parts of the seat like the seat bottom that comes in contact with the back of the thighs.

A respiration sensor can measure aspects of respiration via sensors placed on the seat belt to sense the movement of the chest, or located in the seat, or using a distance sensor that is located in front of the occupant pointing at the occupant's chest.

A temperature sensor can measure the temperature of the occupant using skin sensors. One option would be to use multiple skin sensors in several locations to try to improve the accuracy. A thermal imaging sensor pointed at the skin could provide temperature measurements of the occupant.

A blood oxygen sensor can measure blood oxygen saturation levels using a sensor mounted close to the seat where the occupant can place a finger, a sensor mounted where the occupant touches the steering wheel, or a sensor mounted where the occupant touches the seat or other part of the vehicle.

A blood pressure sensor can measure blood pressure using a wrist cuff.

A blood sugar sensor can measure blood sugar levels by using sensors mounted on the side of the seat or in the central console. The sensor could be non-invasive or may require a tiny lancet to sense the blood sugar level from peripheral capillary blood.

A blood alcohol sensor can measure blood alcohol levels with sensors mounted on a breathing tube attached to the side of the seat, or non-invasive sensors placed high on the seat belt could sense the levels from exhaled air.

A brain wave sensor can measure brain activity for an electroencephalogram tracing with sensors mounted on or near the skull and face, like a cap of sensors.

A video sensor could capture the occupant on video and could provide video to analyze and provide information about health parameters, for example, respiration rate or pulse rate.

The health-monitor concierge 10 could monitor health parameters, such as, weight, height, cardiac signals, pulse rate, electrocardiographic tracings, respiration rate, temperature, oxygen saturation levels, blood pressure, blood sugar, blood alcohol, electroencephalogram tracings, body fat content, body mass index, metabolic rate, cardiac arrhythmias, dyspnoea, cholesterol levels, triglyceride levels and ratios, ovulation predictions, and other health parameters.

The health-monitor concierge 10 could report pulse rate, electrocardiographic tracings and cardiac arrhythmias identified by analyzing the cardiac sensor signal.

The health-monitor concierge 10 could check the occupant's height against the other information gathered during the registration process.

The health-monitor concierge 10 could report the occupant's temperature from temperature sensor data by averaging from multiple temperature sensors, like multiple skin temperature sensors, if necessary.

The health-monitor concierge 10 could report the occupant's body fat content by calculating it from the body weight sensor input.

The health-monitor concierge 10 could report the body mass index, by calculating it from the occupant's height and weight.

The health-monitor concierge 10 could report the metabolic rate, calculating it from measured biological parameters and/or profile information, from the occupant's registration.

The health-monitor concierge 10 could report dyspnoea, shortness of breath, from the respiratory sensor signal.

The health-monitor concierge 10 could report cholesterol levels, cholesterol ratio, triglyceride levels and triglyceride ratios from the blood drawn with the tiny lancet used to sample blood for blood sugar levels.

The health-monitor concierge 10 could analyze the video signal of the occupant to pick up respiration information, pulse rate, or other biological parameters.

The health-monitor concierge 10 could analyze the video signal or other gathered health parameters to detect epileptic seizures and epileptic movements.

The health-monitor concierge 10 could analyze the gather health parameters like temperature and other health parameters to predict ovulation.

The health-monitor concierge 10 could report on proper posture.

### Embodiment Two

FIG. 2 is a block diagram of a second embodiment of a health-monitor concierge 34. In this embodiment, the data-gathering unit 12 from FIG. 1 can send the measured biological data and monitored health parameters periodically to a central data repository 30. The data-gathering unit 12 can send the data via either the long-range communication device 20 or the short-range communication device 18. For those occupants who require continuous monitoring, the long-range communication device 20 would be preferred because it can transmit data in real time to the central data repository 30. If the short-range communication device 18 is used, then the data would be relayed by another communication device, for example, a cell phone, that sends the data to the central data repository 30.

The data-gathering unit 12 can accumulate the collected data and send it in batch to the central repository 30 or the data-gathering unit 12 could stream the data continuously to the central repository 30. The vehicle can have an integrated data-gathering unit 12 that can buffer the data (measured biological parameters or measured health parameters) locally, and can send the data to the central repository 30 on turning off or starting the vehicle.

The central data repository 30 receives data sent from the data-gathering unit 12 and stores the data. The central data repository 30 can make the data available to various applications for the occupant, depending on the occupant profile. The data will also be stored for later analysis or access, by download or other means. From the central data repository 30 the data can go to a number of destinations including a personal health record 40, a physician record 38, a monitoring station 32, or other destinations.

The personal health record 40 is a place for an individual to stores their health information, for example, Microsoft's HealthVault, Patientslikeme.com, other electronic personal health records, or non-electronic personal health records. For non-electronic records, the health-monitor concierge can send the information by faxing, or printing and mailing the information.

The physician record 38 is a system that stores a doctor's patient information, if the physician has an Electronic Medical Record (EMR) system, then the central data repository 30 may be able to send the information directly to the EMR. If the physician does not have an EMR that can receive electronic communications, then the health-monitor concierge 34 can fax or print and mail the information to the occupant's physician for entry into the EMR system or for filing in the physical Medical Records. Similarly, the information could be sent to a local Health Information Exchange (HIE), an Accountable Care Organization (ACO), a weight loss program like Weight Watchers, Emergency Medical Responder, a medical specialist they are seeing, a smart health card or other destination.

The health-monitor concierge 34 can monitor health parameters with the monitoring station 32, as specified by the thresholds in the occupant profile, and the health-monitor concierge 34 can notify the occupant, occupant's physician, other interested parties or other systems when an alert is triggered by a threshold being passed. In another embodiment, the computing device 22 monitors the thresholds from the profile. The profile can specify to monitor and provide real-time immediate feedback to the occupant or to buffer the data and send it off periodically to the central data repository 30 for the monitoring station 32 to analyze later.

If real time monitoring is desired then the data would not be buffered but instead streamed real time to the central data repository 30 to be monitored by the monitoring station 32, or the thresholds could be monitored on the data-gathering unit 12 in real-time and alerts and notifications could be done locally. The health-monitor concierge 34 can reduce communication costs by buffering the content and sending the data in bursts.

The monitoring station 32 can detect when the measured health parameter passes a threshold and triggers an alert. The health-monitor concierge 34 can notify the occupant, other concerned individual, or other interested systems about the alert. The health-monitor concierge 34 can notify the occupant via the display or relay the alert to the occupant or other interested party such as a doctor, caregiver, parent, employer, insurance company, courts or others. The alerts can be detected at the monitoring station 32 or the data-gathering unit 12.

The monitoring station 32 can analyze the data using tools such as pattern recognition software that look for patterns and aberrations, and set alerts when approaching or exceeding thresholds (the pre-set limits from the profile) and take other actions indicated by the profile including setting and alerting the occupant as customized, and possibly initiating a cascade of pre-programmed activities. Computers and/or people can operate the monitoring station 32.

The data from the central data repository 30 can be analyzed across multiple occupants to find correlations between the data and a medical concern and use that information to find ways to improve the medical concern. For example, the accuracy and services that the health-monitor concierge 34 provides could be improved base on the analysis of the multiple occupants.

The health-monitor concierge 34 can use the measured health parameters to provide relevant information to the occupant. The health-monitor concierge 34 could connect the occupant with a nurse to provide advice or explain to the occupant the measured health parameters. The health-monitor concierge 34 could notify the occupant's physician so the physician could have the information in their records for the occupant's next appointment, or schedule an appointment to discuss the monitored health parameters that generated the alert.

The alert could be an offer of assistance including connecting the occupant to their doctor, a non-emergency health service provider or (especially in the case with real-time monitoring) an emergency health service provider, and if necessary, dispatching emergency personnel to assist the occupant as appropriate. The notification can be done with any number of mechanisms, for example, through the display 14, audio on the device or through the vehicle's speakers, or a phone call. The monitoring thresholds can be stored at the monitoring station 32 or on the data-gathering unit 12.

The health-monitor concierge 34 can publish the measured health parameter, for example, the data could be available on a personal webpage, or shared with other sites including social sites like Facebook, as discussed above.

The health-monitor concierge 34 can provide concierge services, like non-emergency audio links to answer health queries, related or not related to the monitored health parameters. The health-monitor concierge 34 could coordinate visits and help to establish appointments with physician offices, diagnostic centers, and hospitals. The health-monitor concierge 34 can help find a pharmacy in the area that has a prescription item in stock and available, before routing the occupant to that pharmacy. The monitoring station 32 could provide these concierge services, or some of these services could be provided by or through the data-gathering unit 12.

The data-gathering unit 12 could have the capability of voice communication with the monitoring station 32.

The health-monitor concierge 34 could charge a reoccurring fee for the monitoring of biological parameters.

The health-monitor concierge 34 could help statistically study a population of occupants. For example, the health-monitor concierge 34 could help determine statistics like the average heart rate for the population of people on public transportation, and then it could be seen if changes in the environment affect the health statistics of the group.

### Uses for the Health-Monitor Concierge

The health-monitor concierge has a number of uses. The health-monitor concierge could monitor the health of individuals entrusted with critical functions, such as airline pilots, bus drivers, truck drivers and others. The health-monitor concierge can monitor multiple health parameters of an occupant of the vehicle, alert them to any aberrations so that early intervention can occur. For example, if a heart attack is detected in an airplane pilot, then the co-pilot could be signaled of the emergency. If the driver of a bus is having a heart attack perhaps the bus could be brought to a controlled stop.

The health-monitor concierge can monitor and report on the effectiveness of any on-going clinical intervention in real time without waiting for follow up visits to the doctor's office. The health care system can use the health-monitor concierge to monitor remotely the progress and would have data, trends and patterns to aid in the diagnosis, treatment and adjustments to treatment for the occupant. The health-monitor concierge could reduce the time to arrive at a diagnosis and enable early initiation of treatment, significantly reducing morbidity in populations. The health-monitor concierge can also serve to encourage individuals who are making efforts to modify their lifestyles to be healthier by providing real time information on the results of their efforts. By using the health-monitor concierge one can intervene in the early stages of any disease and monitoring can reduce the severity of the disease.

The health-monitor concierge can be used by companies with fleets of vehicles to monitor the health and alertness of their drivers. So any company with a fleet of vehicles: package delivery like FedEx or UPS; transportation like airlines, buses, or trains; trucking companies operating on the highway for long distances; taxi companies; snow plow fleets like a municipality may have; or local delivery companies that provided bottled drink and produce to local stores. Any situation where drivers are either in the vehicle for long periods of time or are in and out of their vehicles throughout the day could find the continuous health care monitor concierge useful.

The health-monitor concierge can even be useful in a non-vehicle setting for monitoring employees, especially those in critical rolls such as air traffic controllers, or just to monitor those sitting in desks at work for health conditions.

The health-monitor concierge could be used to track impact of health interventions or health programs on a population, be it bus rider, train riders, taxi cab drivers, or just employees. The information could be gathered up just on the occupant, without identifying the individual occupying the seat. The information could be aggregated up to inform health policy for the government, or help the insurance industry (life, health, or auto) to ascertain risks and or tailor intervention strategies.

The health-monitor concierge could be used by courts to monitor a vehicle driver for compliance with court orders.

The health-monitor concierge could be used on airplane seats to provide occupants with health parameter monitoring. The occupant could be identified by their frequent flyer ID and the health parameter data could be associated to the occupant so overtime the occupants health could be monitored.

The health-monitor concierge could be used in public transportation and the occupant could be identified by their credit card or other information like their name. The health-monitor concierge then finds the occupants profile and records and monitors their health parameters as their profile indicates.

Where there has been a vehicle crash, the health-monitor concierge could be used by first responders to get triage information to decide which accident victims should be prioritized. The data gathered by the health-monitor concierge can be used like an airplane black-box to get information about the situation leading up to the crash, providing forensic information that could be used in legal settings.

Although some descriptions include steering wheel or other indications that the health-monitor concierge is for a driver, the health-monitor concierge can be use for any occupant of a vehicle, like passengers.

It is to be understood that although certain functionality has been identified as residing in the data-gathering unit 12, the central data repository 30 or the monitoring station 32 the functionality can, in practice, be located in any number of areas, like the data-gathering unit 12, the central data repository 30, the monitoring station 32 or other appropriate locations capable of carrying out the described functionality. For example, although the calculation and analysis of health parameters can be described as being done on the data-gathering unit 12 it could also be done at the monitoring station 32.

The two health-monitor concierges (10 and 34) the alternatives discussed above are merely illustrative of the best modes to create a health-monitor concierge. Many alternative approaches would be apparent to those of skill in the art upon reading the above description. This description should be understood to include all novel and non-obvious combinations of the elements described, and claims may be presented in this or a later application to any novel and non-obvious combination of these elements. The scope of the invention is determined, not with reference to the above description, but in reference to the appended claims.

Give all terms used in the claims their broadest reasonable construction and their ordinary meaning as understood by those skilled in the art. Use of the singular articles such as “a”, “the”, “said”, etc. should be read to recite one or more of the indicated elements.

What is claimed is:

1. A system for personal health monitoring comprising:
a profile creator that includes a list of medical concerns where each medical concern is associated to a set of health parameters to monitor;
an occupant profile that contains a medical concern from the list of medical concerns and includes a threshold corresponding to at least one of the health parameters associated to the set of medical concerns;
a data-collection unit that receives biological signals from a vehicle occupant from sensors mounted in the vehicle;
a memory device for storing the received biological signals;
a data monitor that provides measured health parameters using the biological signals and sets an alert when the measured health parameter passes the corresponding threshold; and
a notification mechanism that notifies an interested party when the alert occurs.

2. The system of claim 1 wherein the memory device is part of the data-collection unit.

3. The system of claim 1 wherein the memory device is part of a central repository.

4. The system of claim 3 wherein the data is available on a web page and is shared with other websites.

5. The system of claim 1 wherein the medical concern is becoming pregnant and the alert is predicting ovulation.

6. The system of claim 1 wherein the data monitor aggregates data from a group of occupants.

7. The system of claim 6 wherein the group of occupants all have the same medical concern and the analysis is used to improve the monitoring of the medical concern.

8. The system of claim 1 wherein the interested party is the employer of the occupant and the employer has a fleet of vehicles.

9. The system of claim 1 further comprising an appointment scheduler that helps the occupant establish an appointment related to a monitored concern.

10. The system of claim 1 wherein the interest party is a doctor and the notification informs the doctor of the effectiveness of a treatment.

11. A method for personal health monitoring comprising:
running a profile creator that includes a list of medical concerns where each medical concern is associated to a set of health parameters to monitor, where the running of the profile creator creates an occupant profile that contains a medical concern from the list of medical concerns and includes a threshold corresponding to at least one of the health parameters associated to the set of medical concerns;
receiving biological signals from a vehicle occupant from sensors mounted in the vehicle;
storing the received biological signals;
providing measured health parameters using the biological signals;
setting an alert when the measured health parameter passes the corresponding threshold; and
notifying an interested party when the alert occurs.

12. The method of claim 11 wherein storing the received biological signals is done at a data-collection unit.

13. The method of claim 11 wherein storing the received biological signals is done at a central repository.

14. The method of claim 13 further including providing the biological signals on a web page and sharing the biological signals with other websites.

15. The method of claim 11 wherein the medical concern is becoming pregnant and the alert is predicting ovulation.

16. The method of claim 10 further including aggregating data from a group of occupants.

17. The method of claim 16 wherein the group of occupants all have the same medical concern and the analysis is used to improve the monitoring of the medical concern.

18. A system for personal health monitoring comprising:
means for running a profile creator that includes a list of medical concerns where each medical concern is associated to a set of health parameters to monitor, where the means for running a profile creator creates an occupant profile that contains a medical concern from the list of medical concerns and includes a threshold corresponding to at least one of the health parameters associated to the set of medical concerns;
means for receiving biological signals from a vehicle occupant from sensors mounted in the vehicle;
means for storing the received biological signals;
means for deriving measured health parameters using the biological signals and setting an alert when the measured health parameter passes the corresponding threshold; and
means for notifying an interested parties when the alert occurs.

19. The system of claim 18 further comprising a means for providing the biological signals on a webpage and sharing the biological signals with other websites.

20. The system of claim 18 further comprising a means for aggregating and analyzing data from a group of occupants, where the group of occupants all have the same medical concern and the analysis is used to improve the monitoring of the medical concern.
